(19) United States
(12) Patent Application Publication
(10) Pub. No.: US 2008/0076989 A1
(43) Pub. Date: Mar. 27, 2008

(54) Tail Mounting Clip for Securely Mounting Sensor to Tail and a Tail Mounted Pulse Oximetry Sensor System Using Same

(75) Inventors: Bernard F. Hete, Kittanning, PA (US); Eric W. Starr, Allison Park, PA (US)

Correspondence Address: BLYNN L. SHIDELER;THE BLK LAW GROUP, 3500 BROKKTREE ROAD, SUITE 200, WEXFORD, PA, 15090, US

(73) Assignee: STARR Life Sciences Corp., Oakmont, PA (US)

(21) Appl. No.: 11/858,874

(22) Filed: Sep. 20, 2007

Related U.S. Application Data
(60) Provisional application No. 60/826,479, filed on Sep. 21, 2006.

(51) Int. Cl. A61B 5/1455; A44B 21/00

(52) U.S. Cl. 600/323; 24/331; 600/344

(57) ABSTRACT

A tail mounted clip adapted to receive one or more sensors thereon that incorporate at least one circular groove on a tail engaging face thereof for locating the clip on the tail of a subject and retaining clip and associated sensor(s) properly in place. This configuration allows the clip to be securely located in any circumferential orientation around the tail of the subject. The present invention will have the sensors properly positioned adjacent the tail and will prevent relative movement between the sensors and the tail. The tail mounting clip is used to hold an LED sensor and photodiode for making pulse-oximetry measurements, also called a photoplethysmographs.

### RELATED APPLICATION

This application claims the benefit of provisional patent application Ser. No. 60/826,479 filed Sep. 21, 2006 entitled “Tail Mounting Clip for Securely Mounting Sensor to Tail and Tail Mounted Pulse Oximetry Sensor System Using Same.”

### BACKGROUND OF THE INVENTION

1. Field of the Invention

The present invention relates to a clip for mounting a sensor to the tail of an animal, such as for use in research applications. More particularly the present invention relates to tail mounting clips for securely mounting physiologic sensors, such as pulse oximetry sensors, onto the tail of rodents and small mammals.

2. Background Information

In conducting research on animals, particularly small mammals, such as most commonly mice, a researcher must spend time and money reviewing and implementing the data collection methods and devices that will be required. Existing devices are often not well-suited for these applications, which can compromise or prevent data collection. For example, consider that the shape of most animal tails is cylindrical. When applying a sensor to the tail of a rodent or other small animal, it can be difficult to attach the sensor utilizing a conventional sensor mounting clip that is designed for other, non-rounded appendages of subjects (i.e. human earlobes). Researchers have experienced that when using the current sensor mounting clips on the tail of the rodent subject, the sensor clip would not stay located properly, and accurate and reliable measurements could not be obtained.

The problem is exacerbated by the fact that some research effectively requires measurements to be taken at the tail of the subject. The tail of the subject often provides a convenient place for sensor attachment from the standpoint of non-interference between the sensor and the test being conducted on the animal. Further, the tail is a convenient place for sensor placement from the standpoint of the convenience to the researcher. Consequently there remains a need in the industry for a secure, fast attachment of sensors, particularly physiologic sensors, to the tails of subjects, such as rats and mice.

### SUMMARY OF THE INVENTION

The present invention provides a clip adapted to receive one or more sensors thereon that incorporate at least one circular groove on a tail engaging face thereof for locating the clip on the tail of a subject and retaining the clip and associated sensor(s) properly in place. This configuration allows the clip of the present invention to be located in any circumferential orientation around the tail of the subject. The present invention will have the sensor properly positioned adjacent the tail and will prevent relative movement between the sensor and the tail. The present invention is designed for the tail of a subject, but, of course, the sensor could work effectively on other animal appendages, even if the other appendage does not fit within the defined groove of the clip. Where the present clip is attached to the subject on a location of the subject that is not received within the groove, at least partially, the clip will effectively operate in the same manner as prior art sensor attaching clips.

In one non-limiting embodiment, the tail mounting clip is used to hold an LED sensor and photodiode for making pulse-oximetry measurements, also called a photoplethysmograph. These sensor systems typically incorporate an incandescent lamp or light emitting diode (LED) to trans-illuminate an area of the subject, e.g. an appendage, that contains a sufficient amount of blood. The light from the light source disperses throughout the appendage, which can be considered as broken down into non-arterial blood components, non-pulsatile arterial blood and pulsatile blood. The light detector, such as a photodiode, is placed on the opposite side of the appendage to record the received light. Due to the absorption of light by the appendage's tissues and blood, the intensity of light received by the photodiode is less than the intensity of light transmitted by the LED (or other light source). Of the light that is received, only a small portion (that effected by pulsatile arterial blood), usually only about two percent of the light received, behaves in a pulsatile fashion. The beating heart of the subject and the breathing of the subject combine to create this pulsatile behavior. The “pulsatile portion of light” is the signal of interest and effectively forms the photoplethysmograph. The absorption described above can be conceptualized as AC and DC components. The arterial vessels change in size with the beating of the heart. The change in arterial vessel size causes the path length of light to change from dₘᵢₙ to dₘₐₓ. This change in path length produces the AC signal on the photo-detector, I_{L}, to I_{H}. The AC Signal is, therefore, also known as the photo-plethysmograph.

The absorption of certain wavelengths of light is also related to oxygen saturation levels of the hemoglobin in the blood transfusing the illuminated tissue. In a similar manner to the pulse monitoring, the variation in the light absorption caused by the change in oxygen saturation of the blood allows for the sensors to provide a direct measurement of arterial oxygen saturation where two light sources of differing wavelengths are used, and when used in this context the sensors are known as oximeters. The use of such sensors for both pulse monitoring and oxygenation monitoring is known and in such typical uses the devices are often referred to as pulse oximeters. These devices are well known for use in humans and large mammals and are described in U.S. Pat. Nos. 4,621,643; 4,700,708 and 4,830,014 which are incorporated herein by reference. Pulse Oximetry sensors for small mammals are available under the Mouse Ox™ brand from Starr Life Sciences, Inc.

The present clip allows pulse oximetry sensors to be securely attached to the tail of the subject, particularly rodents such as mice and rats and other small mammals. The present invention is particularly well suited for the application of such pulse oximeters onto the tails of subjects. One embodiment of the present invention provides a groove on the tail engaging face of a first body portion with the LED transmission source coupled thereto and designed to diffuse through a translucent part of the first body portion of the coupler. This structure is believed to maximize the light transmitted to and through the tail. One embodiment of the present invention provides a groove on the tail engaging face of a second body portion of the coupler with the photo-detector or receivers coupled thereto and designed to minimize ambient light received thereby. The second body portion may include an aperture aligned with the groove to maximize light received that is transmitted through the tail. The receipt of the tail in the groove of the second body will further assure that the light transmitted through the tail is received by the detector. Further, the second body may be formed of light-blocking material (e.g. coated so as to not be translucent) to prevent the photo detectors from receiving too much ambient light that dilutes the signal.

Although the present clip is particularly well suited for pulse oximeters as discussed above, it can be used effectively for many sensors. Essentially, it could be used for any application in which a sensor is needed to clip onto the tail of a subject. Temperature sensors, position sensors, blood pressure monitors are some of many examples.

In one non-limiting embodiment of the present invention, the clip is a spring-loaded, pivoted body-type clamp, but it could be attached with some other method, including adhesives, magnetic elements, tape, etc. The illustrated embodiments discussed below also possess the rounded, transverse slot on both halves of the clip, but it could be done on only one half. Additionally, the slot could have a variable cross-sectional shape, and does not have to be limited to semi-circular. It could also be V-groove, or square in cross-section. Although our sensor prototype uses a groove transverse to the direction of the clip, it could also run axially with the clip, or at any angle between.

In one non-limiting embodiment of the invention a tail restraining device for supporting one or more sensors on an animal tail comprises a tail mounting clip having at least one tail engaging face; at least one groove provided on the tail engaging face configured to partially receive at least a portion of said animal tail in the groove and for locating and retaining said device and one or more sensors in said operative orientation on said animal's tail. The device may includes a pair of opposed tail engaging faces on opposed body portions of the clip and further including retaining arms which are adapted to couple said one or more sensors to the body portion in a position aligned with the groove. The first body portion may be formed of a translucent material whereby the first body portion is designed to diffuse a light signal. The second body portion may be formed of an opaque material that prevents light transmission therethrough, and wherein the second body portion may include an aperture aligned with the groove and with the sensor that is attached thereto. The one or more sensors may further comprise pulse oximetry sensors.

In one non limiting embodiment of the invention a pulse oximetry sensor system comprises a mounting device including a plurality of body portions, whereby at least two of said body portions define a subject engaging face having at least one groove thereon extending transversely across at least a portion of said subject engaging face, and a pulse oximetry transmitter sensor coupled to one body portion of said device aligned with said groove, and a pulse oximetry receiver sensor coupled to another body portion of said device aligned with said transmitter to receive a signal therefrom. The groove in the first body portion may extend to about ⅔ of the depth of the body portion at the location of the groove. The clip may include a transverse circular groove on the engaging face of a second body portion.

One non-limiting embodiment of the invention may provide a pulse oximetry sensor system comprising a mounting device including a plurality of body portions, whereby at least two of said body portions define a subject engaging face, a pulse oximetry transmitter sensor coupled to a translucent body portion of said device wherein said translucent body portion diffuses a light signal from said transmitter; and a pulse oximetry receiver sensor coupled to an opaque body portion of said device to receive a signal therefrom through an aperture provided in said opaque body portion. The clip may be a spring-loaded, pivoted body-type clamp. The clip may include a groove on at least one subject engaging face, wherein the groove is configured to partially receive at least a portion of the subject's tail therein for locating the clip on the tail of a subject and for retaining the clip and associated sensors properly in place on the tail of the subject. The transmitter portion and the receiver portion may be aligned with the groove. Retaining arms may be provide which are adapted to couple the transmitter portion and the receiver portion to respective body portions in a position aligned with the groove. The system may further include a spring biasing the body portions to a closed position.

One non limiting embodiment of the present invention may provide a minimal trauma tail mounting device comprising a pair of pivotably mounted body portions having a tail engaging face incorporating at least one transverse groove extending across each tail engaging face, wherein said grooves are aligned and configured to receive a subject's tail therein for locating the device on the tail of said subject and for retaining said device properly in place on said tail of said subject, and a spring biasing the body portions toward a closed position in which the tail engaging faces are adjacent each other. The clip may be configured to receive pulse oximetry sensors. Retaining arms may be provided which are adapted to couple the transmitter portion and the receiver portion of the pulse oximetry sensors to respective body portions in a position aligned with the groove.

One non-limiting aspect of the invention provides a tail mounting clip comprising a pair of body portions pivoted together and each body portion including a tail engaging face, and a spring biasing the body portions toward a closed position in which the tail engaging faces are adjacent each other, wherein the spring exerts a relatively constant closing force over the operative range of the body portions. Each engaging face may incorporate at least one transverse groove extending across the tail engaging face, wherein the grooves on the respective tail engaging faces are aligned and are configured to receive the subject's tail therein for locating the clip on the tail of a subject and for retaining the clip properly in place on the tail of the subject. The clip may be configured to receive pulse oximetry sensors.

These and other advantages of the present invention will be clarified from the attached figures wherein like reference numerals represent like elements throughout.

### BRIEF DESCRIPTION OF THE FIGURES

FIG. 1 is a side view of a tail mounting clip for securely mounting physiologic sensors, such as pulse oximetry sensors, onto the tail of rodents and small mammals in accordance with one embodiment of the present invention;

FIG. 2 is a side view of the tail mounting clip of FIG. 1 with the sensors removed for clarity;

FIG. 3 is a plan view of the pulse oximeter receiver mounting side of the tail mounting clip of FIG. 1 with the sensors removed for clarity;

FIG. 4 is a schematic side view of the pulse oximeter receiver mounting side of the tail mounting clip of FIG. 3 with the sensors removed for clarity;

FIG. 5 is a schematic section view of the pulse oximeter receiver mounting side of the tail mounting clip of FIG. 4 with the sensors removed for clarity;

FIG. 6 is a plan view of the pulse oximeter transmitter mounting side of the tail mounting clip of FIG. 1 with the sensors removed for clarity;

FIG. 7 is a schematic side view of the pulse oximeter transmitter mounting side of the tail mounting clip of FIG. 3 with the sensors removed for clarity;

FIG. 8 is a side view of the tail mounting clip of FIG. 1 for securely mounting physiologic sensors, such as pulse oximetry sensors, onto the tails of rodents and small mammals; and

FIGS. 9-10 are perspective views of the tail mounting clip of FIGS. 1 and 8 for securely mounting physiologic sensors, such as pulse oximetry sensors, onto the tail of a subject rodent.

### DESCRIPTION OF THE PREFERRED EMBODIMENT

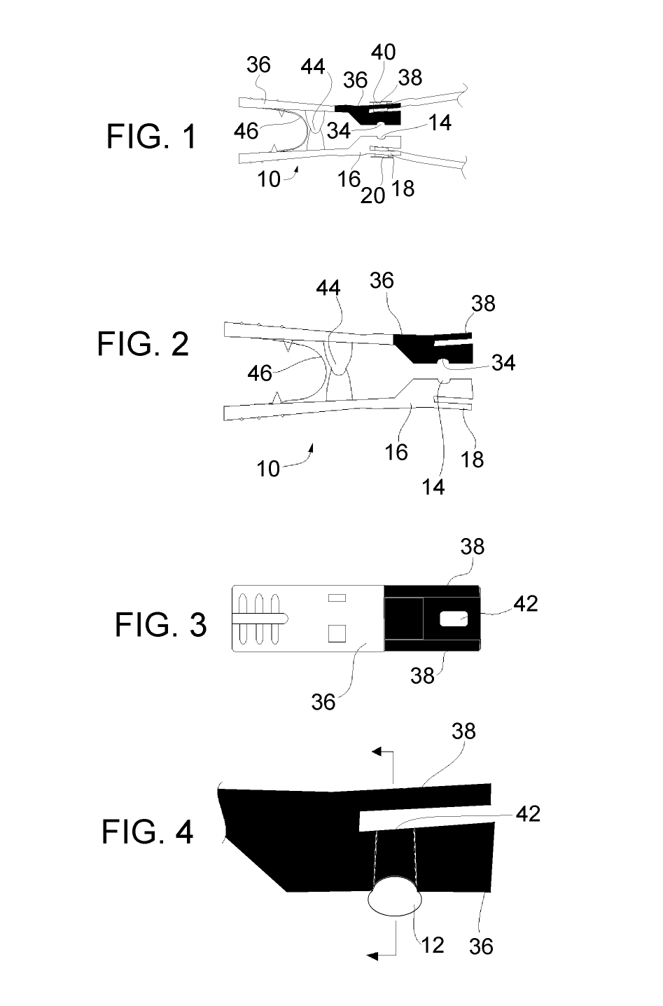

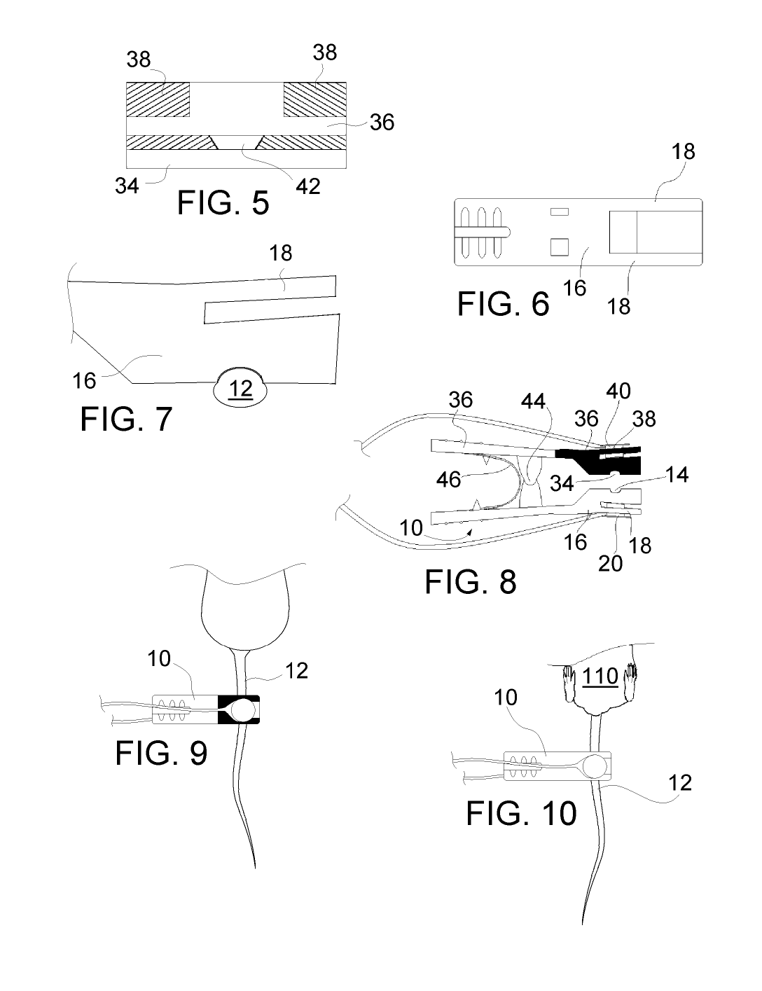

FIGS. 1-10 illustrate a tail mounting clip 10 for securely mounting physiologic sensors, such as pulse oximetry sensors, onto the tail 12 of rodents and small mammals in accordance with one embodiment of the present invention. The term clip within the meaning of this application is a small fastener used to hold articles together, namely physiologic sensors onto a subject. Within the meaning of the present invention, both the transmitters and receivers of pulse oximetry sensors will be broadly considered sensors. A wide variety of sensors can be used by researchers, such as temperature sensors, resistance sensors, pressure sensors, magnetic sensors. The clip 10 is particularly well suited for use with sensors that transmit signals, e.g. light, through the subject tail to generally aligned receiving sensors opposite there from.

The clip 10 of the present invention as shown in the figures provides a transverse circular groove 14 on the tail engaging face of a first body portion 16. The first body portion 16 includes retaining arms 18 whereby the LED transmission source 20 of pulse oximetry sensors can be easily coupled thereto. The use of the retaining arms 18 is known in the art for coupling associated sensors, such as a transmitter 20. The groove 14 is circular in cross-section and extends generally perpendicular across the body portion 16. The groove is preferably sized to accommodate a conventional subject's tail 12 as shown schematically in FIG. 7. The groove may extend to about ⅔ of the depth of the body portion at the location of the groove leaving about ⅓ for the structural integrity of the body portion 16. The body portion is preferably formed of a translucent plastic material whereby the body portion 16 is designed to diffuse the light from the LED transmission source 20 (which will have two LED sources of different wavelengths as known in the art) and transmit the diffused light through the adjacent tail 12 of the subject. This structure is believed to maximize the light transmitted to and through the tail 12 and to properly align the sensors 20 with the tail. The body portion 16 may be easily formed by injection molding or the like as is known in the art. Other translucent material other than plastic may be used, if desired. If a non-translucent material is used, then a transmission aperture would need to be provided to allow for the light to be transmitted through the tail, where the clip is used for light-based applications.

The embodiment of the clip 10 of the present invention provides a transverse circular groove 34 on the tail engaging face of a second body portion 36. The second body portion 36 includes retaining arms 38 whereby the photo-detectors 40 of pulse oximetry sensors can be easily coupled thereto. The use of the retaining arms 38 is known in the art for coupling associated sensors, such as photo-detectors 40. The groove 34 is circular in cross-section and extends generally perpendicularly across the body portion 36 and generally opposed from groove 14 of body portion 16.

The groove 34, like groove 14, is preferably sized to accommodate a conventional subject's tail 12 as shown schematically in FIG. 4. The groove 34 may extend to about ⅔ of the depth of the body portion 36 at the location of the groove 34 leaving about ⅓ for the structural integrity of the body portion 36. The body portion 34 is preferably formed of, or coated with, a non-translucent or opaque plastic material and designed to minimize ambient light received by the receivers 40. The second body portion 36 may include an aperture 42 aligned with the groove 34 to maximize light received that is transmitted through the tail 12. The receipt of the tail 12 in the groove of the second body portion 36 will further assure that the light transmitted through the tail 12 is received by the detector 40. Further, with the second body portion 36 formed of light-blocking material (e.g. coated so as to not be translucent or opaque), the second body portion 36 serves to prevent the photo detectors 40 from receiving too much ambient light that dilutes the signal. The non-translucent configuration with the aperture 42 is believed to help reduce signal noise in the resulting device. The proper location and orientation of the sensors (20 and 40) relative to the appendage (the tail) is also believed to reduce noise in the resulting signal. The grooves 14 and 34 will receive the appendage or tail therein, thereby properly locating and aligning the appendage (e.g. tail) and the clip. The coating of the second body portion 36 with a non-translucent material results in another aspect of the invention in which the sensors 20 and 40 are effectively color-coded to be received in the proper body portion 16 and 36, so that the device is properly assembled. This minimizes the likelihood of the researchers flipping the location of the transmitter 20 and receiver (collectively “sensors”).

In the illustrated but non-limiting embodiment of the present invention, the clip 10 is a spring-loaded, pivoted body type clamp, wherein the first body portion 16 and the second body portion 36 include interconnection pivot-forming elements 44 defining a pivot axis with a spring member 46 biasing distal ends of the first and second body portions away from each other such that the grooves 14 and 34 are biased toward each other and are biased into engagement with the tail 12. The spring member may be a leaf spring. It is contemplated that a leaf spring that is attached at only one body portion 16 or 36, and which “floats” relative to the opposed body portion, can provide a more constant force over a relatively large range of motion. Providing a relatively constant force over a wide variety of motion allows the clip 10 to be suitable for a wide effective range of tails, e.g. ⅛″ to about ¾″ diameter tails. If the variation in force becomes too wide over an effective range, the clip 10 may require calibration for the various tail diameters. Within the operative range of ⅛″ to about ¾″ diameter tails, the relatively constant force of the spring will be within about 50% of the maximum value of compression force within the range. Consequently, the phrase “relatively constant force” relating to spring force within the meaning of this application is that the spring force will vary less than about 50% of the maximum value of compression force within the operative range. As a comparison, the operative range reflects a diameter change of six times, and linear springs would expect a variance of six times over the same range (i.e. the minimum force for the linear spring would be about ⅙ of the maximum force over the operative range).

Another constant force clip-holding technique is to remove the spring and use or add a given hold-down weight to the upper body portion (16 or 36, depending on orientation) after the clip 10 is in position. This would require that the tail 12 is otherwise restrained and that the orientation of the clip allows for an added weight. The “upper” body (16 or 36) could include a weight-retaining member such as a post that the hold-down weight is added onto. Either body portion could be designated as the “upper” body portion.

The body portions 16 and 36 could be attached using further alternative attachment methods, including adhesives, magnetic elements, tape, hold-down straps, elastomeric bands, or combinations thereof without, departing from the scope of the present invention.

The illustrated embodiment also possesses the rounded, transverse groove 14 and 34 on both body portions 16 and 36 (i.e. halves) of the clip 10, but a single semi-circular tail receiving groove 14 or 36 could be done on only one half. Additionally, the groove 16 or 36 could have a variable cross-sectional shape, and does not have to be limited to semi-circular. It could also be a V-groove, or square in cross-section. The illustrated embodiment uses grooves 14 and 34 transverse to the direction of the clip 10, but it could also run axially with the clip, or at any angle between.

FIG. 8 is a perspective view of the tail mounting clip 10 for securely mounting physiologic sensors, particularly pulse oximetry sensors, onto the tails of rodents and small mammals. FIGS. 9-10 are perspective views of the tail mounting clip 10 securely mounting physiologic sensors, such as pulse oximetry sensors, onto the tails 12 of subject rodents. Further, the user then need only clamp the clip 10 around the subject's tail 12, which is a mouse in FIGS. 9-10, placing the tail at least partially within the grooves 14 and 34 to securely position the sensors 20 and 40. For optimal signal with sensors 20 and 40, the clip 10 should be placed on the tail 12 nearest the torso of the rodent (i.e. the thickest part of the tail). The clip 10 of the present invention provides a non-traumatic tail restraint that can be for merely tail restraining purposes without sensors, if desired by the researcher.

It should be apparent that there are many variations to the present invention that can be found within the spirit and scope of the present invention. The apparatus is intended to be defined by the appended claims and equivalents thereto.

What is claimed is:

1. A tail restraining device for supporting one or more sensors on an animal tail comprising:
a tail mounting clip having at least one tail engaging face;
at least one groove provided on said tail engaging face configured to partially receive at least a portion of said animal tail in said groove and for locating and retaining said device and one or more sensors in said operative orientation on said animal's tail.

2. The tail restraining device of claim 1 wherein the device includes a pair of opposed tail engaging faces on opposed body portions of the clip and further including retaining arms which are adapted to couple said one or more sensors to the body portion in a position aligned with the groove.

3. The tail restraining device of claim 2 wherein a first body portion is formed of a translucent material whereby the first body portion is designed to diffuse a light signal.

4. The tail restraining device of claim 3 wherein the second body portion is formed of an opaque material that prevents light transmission therethrough, and wherein the second body portion includes an aperture aligned with the groove and with the sensor that is attached thereto.

5. The tail restraining device of claim 4 wherein said one or more sensors further comprise pulse oximetry sensors.

6. A pulse oximetry sensor system comprising:
a mounting device including a plurality of body portions, whereby at least two of said body portions define a subject engaging face having at least one groove thereon extending transversely across at least a portion of said subject engaging face;
a pulse oximetry transmitter sensor coupled to one body portion of said device aligned with said groove; and
a pulse oximetry receiver sensor coupled to another body portion of said device aligned with said transmitter to receive a signal therefrom.

7. The system of claim 6 wherein the groove in the first body portion extends to about ⅔ of the depth of the body portion at the location of the groove.

8. The system of claim 7 wherein the clip includes a transverse circular groove on the engaging face of a second body portion.

9. A pulse oximetry sensor system comprising:
a mounting device including a plurality of body portions, whereby at least two of said body portions define a subject engaging face;
a pulse oximetry transmitter sensor coupled to a translucent body portion of said device wherein said translucent body portion diffuses a light signal from said transmitter; and
a pulse oximetry receiver sensor coupled to an opaque body portion of said device to receive a signal therefrom through an aperture provided in said opaque body portion.

10. The pulse oximetry sensor system according to claim 9 wherein the clip is a spring-loaded, pivoted body-type clamp.

11. The pulse oximetry sensor system according to claim 9 wherein the clip includes a groove on at least one subject engaging face, wherein the groove is configured to partially receive at least a portion of the subject's tail therein for locating the clip on the tail of a subject and for retaining the clip and associated sensors properly in place on the tail of the subject.

12. The pulse oximetry sensor system according to claim 11 wherein the transmitter portion and the receiver portion are aligned with the groove.

13. The pulse oximetry sensor system according to claim 11 further including retaining arms which are adapted to couple the transmitter portion and the receiver portion to respective body portions in a position aligned with the groove.

14. The pulse oximetry sensor system according to claim 11 further including a spring biasing the body portions to a closed position.

15. A minimal trauma tail mounting device comprising:
A pair of pivotably mounted body portions having a tail engaging face incorporating at least one transverse groove extending across each tail engaging face, wherein said grooves are aligned and configured to receive a subject's tail therein for locating the device on the tail of said subject and for retaining said device properly in place on said tail of said subject; and a spring biasing the body portions toward a closed position in which the tail engaging faces are adjacent each other.

16. The minimal trauma tail mounting device of claim 15 wherein the clip is configured to receive pulse oximetry sensors.

17. The minimal trauma tail mounting device according to claim 16 further including retaining arms which are adapted to couple the transmitter portion and the receiver portion of the pulse oximetry sensors to respective body portions in a position aligned with the groove.

18. A tail mounting clip comprising:
A pair of body portions pivoted together and each body portion including a tail engaging face; and
a spring biasing the body portions toward a closed position in which the tail engaging faces are adjacent each other, wherein the spring exerts a relatively constant closing force over the operative range of the body portions

19. The tail mounting clip according to claim 18 wherein each tail engaging face incorporates at least one transverse groove extending across the tail engaging face, wherein the grooves on the respective tail engaging faces are aligned and are configured to receive the subject's tail therein for locating the clip on the tail of a subject and for retaining the clip properly in place on the tail of the subject.

20. The tail mounting clip of claim 18 wherein the clip is configured to receive pulse oximetry sensors.
